(19) United States
(12) Patent Application Publication
(10) Pub. No.: US 2026/0060680 A1
(43) Pub. Date: Mar. 5, 2026

(54) Aorta Aneurysm Anastomosis Device

(71) Applicant: Yong Chee LEE, Shah Alam (MY)

(72) Inventor: Yong Chee LEE, Shah Alam (MY)

(21) Appl. No.: 18/809,329

(22) Filed: Aug. 20, 2024

(51) Int. Cl. A61B 17/11

(52) U.S. Cl. CPC A61B 17/11; A61B 2017/1107; A61B 2017/1132

(57) ABSTRACT

A medical implant device for Anastomosis of both Aorta Proximal and Distal ends is presented. The device is designed to facilitate the connection between the Aorta and the device, with both ends of the device being one size smaller than the Aorta size. The device features grooves and protrusions located about 8 mm from both ends, which are specifically designed to aid in the application of suture loops to tighten the Aorta and block blood flow between the device and the Aorta, respectively. The design of the device aims to simplify the Anastomosis procedure, reducing the time and skill required for the surgeon to perform the procedure, while also improving the overall safety and efficacy of the Anastomosis.

### TECHNICAL FIELD

This device is designed to connect the Aorta Proximal and Distal ends following surgical procedures such as Frozen Elephant Trunk or Stanford Type A or Stanford Type B. Instead of suturing the anastomosis, the device uses grooves and protrusions to facilitate the tightening of suture loops around both ends of the Aorta, creating a secure connection. This method provides an alternative to traditional suturing techniques and offers greater ease and accuracy in connecting the two ends of the Aorta.

### BACKGROUND ART

From the start of cardiovascular surgery, surgeons have been using hand-held sutures to perform Proximal and Distal anastomoses based on the principles of the suture technique. However, in recent years, various new technique and methods have been developed and investigated to establish of Sutureless Vascular Anastomoses, paving the way for the potential widespread use of Sutureless vascular surgery in the future.

Vascular Anastomosis refer to the process of connecting two blood-carrying vessels. The two main types of an Anastomoses are end to end; end to the side. Typically, Vascular Anastomoses are created using conventional suture techniques, which are commonly regarded as “golden standard” in cardiovascular surgery. However, in cases where the arteries are diseased or in surgeries that have limited access or require minimal incisions, hand suturing can be challenging and time-consuming. Such procedures demand considerable technical expertise and effort.

To ensure the swift and dependable recovery of patients undergoing high risk surgery, it is crucial to have feasible, rapid, safe, and reliable Sutureless Techniques available. Therefore, the development of new Sutureless Techniques is essential for improving patient outcomes and advancing the field of cardiovascular surgery.

### SUMMARY OF THE INVENTION

A novel device has been introduced for the creation of a Sutureless Anastomosis, which involves the use of a new connector. This innovativedevice is known as the Aorta Aneurysm Anastomosis Device (AAAD) and it may serve as an alternative treatment for Sutureless Vascular Anastomosis. The device has been developed based on several key factors including the absence of the need for sutures, shorter operation time, improved safety, enhanced reliability, increased patency, and greater stability of the Vascular Anastomosis.

To elaborate, the Aorta Aneurysm Anastomosis Device (AAAD) has the potential to revolutionize the field of surgical procedures for Vascular Anastomosis by eliminating the need for sutures. The use of this device results in shorter surgical procedures which not only saves time but also reduces the risk of complications associated with longer surgeries. Moreover, the device is designed to provide a safe and reliable alternative to traditional suture-based techniques.

Another significant advantage of the Aorta Aneurysm Anastomosis Device is that it ensures improved patency of the Anastomosis. This means that the blood flow through the Anastomosis is less likely to be restricted, resulting in improved healing and recovery for patients. Additionally, the device provides greater stability to the Anastomosis, reducing the risk of leaks or other complications that may occur with suture-based techniques.

Overall, the Aorta Aneurysm Anastomosis Device (AAAD) represents a significant advancement in the field of surgical procedures for Vascular Anastomosis. Its unique design and features offer numerous benefits over traditional suture-based techniques, making it a valuable tool for surgeons and effective treatment option for patients.

The Aorta Aneurysm Anastomosis Device (AAAD) is composed of one cylindrical tube, 40 mm in length with grooves and protrusions on both ends of the device. The AAAD was made of Titanium or Nitinol.

### BRIEF DESCRIPTION OF THE DRAWINGS

FIG. 1 is a partial cross-sectional view of a cylindrical tube for the Aorta Anastomosis Device.

FIG. 2 is a longitudinal section view of a cylindrical tube for the Aorta Anastomosis Device.

FIG. 3 is a schematic view of a cylindrical tube for the Aorta Anastomosis Device.

FIG. 4 is a longitudinal cross section view of a cylindrical tube (2) with outer diameter of Protrusions which seated with blood vessels (3,4). Sutures (7) are used to connect both ends of the blood vessels to prevent any dislodgement.

FIG. 5 is a longitudinal cross sectional view of a cylindrical tube (1) with suture loops (8) used to secure the blood vessels (5,6) to the cylindrical tube (1).

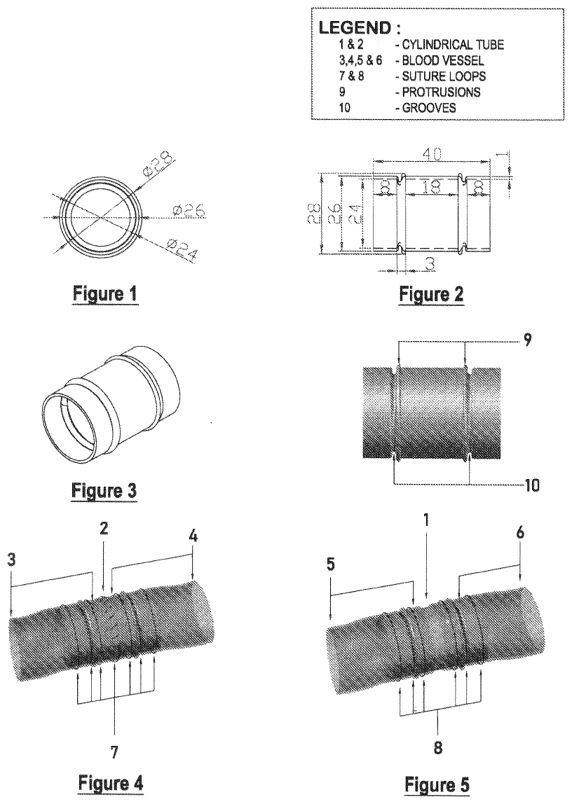

### DESCRIPTION

Introducing the innovative medical implant device for Anastomosis of both Aorta Proximal and Distal. This cutting-edge device is designed to facilitate easy lodging of the Aorta onto the device, with both ends of the device being smaller than the size of the Aorta.

The device is equipped with unique grooves and protrusions, positioned approximately 8 mm from both ends of the device. These grooves are specifically designed to enable the application of suture loops to tightly secure the Aorta to the device, ensuring a secure connection.

The protrusions located adjacent to the grooves are designed to block any blood flow between the device and the Aorta, ensuring the safe and efficient flow of the blood through the Aorta.

This device is a game-changer in the field of medical implants and is sure to revolutionize the way Anastomosis is performed. Its design ensure that the procedure is both efficient and effective, while also being safe for the patient.

1. Aneurysm Anastomosis Device
Cylindrical tube for aneurysm anastomosis, comprising:
1.1 A cylindrical tube (1&2) having a length of 40 mm.
1.2 Grooves and protrusions arranged sequentially along the cylindrical tube (1&2).
1.3 The grooves and protrusions are evenly spaced.
1.4 The grooves and protrusions are positioned at a distance of 8 mm from both ends of the cylindrical tube (1&2).
1.5 The grooves and protrusions serve as markings or indicators for proper alignment during the anastomosis procedure.
1.6 Both ends of the cylindrical tube (1&2) are designed to be smaller than the aorta for easy lodging of the aorta onto the device.
1.7 The protrusions are designed to be larger than the aorta, effectively blocking the blood flow between the device and the aorta.
1.8 The grooves are capable of accommodating 2 to 3 suture loops (7&8) with a depth of 1 mm.
1.9 A suture loop (7&8) is applied before and after the protrusions, each with a height/ radius of 1 mm.
1.10 The device is made of Titanium.
1.11 The device is made of Nitinol.

2. Enhanced Stability
The grooves and protrusions of the cylindrical tube (1&2) according to claim 1 provide increased stability and effectively prevent dislodgement during normal blood flow. Instead of using expandable metallic rings, suture loops (7&8) are applying to grooves and protrusions to prevent dislodgement.

3. Suture Loop Integration
The integration of suture loops (7&8) into the grooves and before and after the protrusions of the device in claim 1 enables secure attachment of blood vessels (3,4,5&6). Without using expandable metallic rings, suture loops(7&8) are used to secure the attachment at grooves and protrusions. Both grooves and protrusions have a radius of≤1 mm.

4. Prevention of Dislodgement
The combination of the cylindrical tube (1&2) design, grooves, protrusions, and suture loops (7&8) as described in claim 1 ensures the prevention of dislodgement of the cylindrical tube (1&2) and blood vessels (3,4,5&6). The depth or radius of grooves and height or radius of protrusions are≤1 mm.

5. Secure Fixation
The suturing of both ends of the blood vessels (3,4,5&6) to the cylindrical tube (1&2) using the sutures described in claim 1 creates a secure and reliable connection. Only suture loops (7&8) are applying to grooves and protrusions.

6. Minimized Risk
The device's design and suturing technique outlined in claim 1 reduce the risk of complications associated with aneurysm anastomosis procedures, including but not limited to leakage or displacement. Leakage or displacement can be secured by hand-tied suture loops (7&8) instead of using clips or metallic rings.

7. Simplified Surgical Procedure
The novel device described in claim 1 simplifies the surgical procedure for aneurysm anastomosis by providing an effective method of securing blood vessels without compromising stability. The design of the grooves is mainly to securely hold the suture loops, while the design of the protrusions is primarily to prevent the dislodgement of the suture loops(7&8).

8. Improved Patient Outcomes
The utilization of the device specified in claim 1 may result in improved patient outcomes by reducing the risk of complications and promoting successful aneurysm repair.

9. Compatibility
The device of claim 1 is designed with a cylindrical body that is compatible with minimally invasive surgical techniques.

10. Biocompatibility
The Aneurysm Anastomosis Device according to claim 1 is equipped with a coating or surface treatment on the cylindrical tube to enhance biocompatibility and reduce the risk of thrombosis.
